(19) United States
(12) Patent Application Publication
(10) Pub. No.: US 2017/0035456 A1
(43) Pub. Date: Feb. 9, 2017

(54) HOLDING MECHANISM AND INSERTION DEVICE

(71) Applicant: OLYMPUS CORPORATION, Tokyo (JP)

(72) Inventor: Ryota KUDO, Sagamihara-shi (JP)

(73) Assignee: OLYMPUS CORPORATION, Tokyo (JP)

(21) Appl. No.: 15/296,246

(22) Filed: Oct. 18, 2016

Related U.S. Application Data
(63) Continuation of application No. PCT/JP2015/075208, filed on Sep. 4, 2015.

(30) Foreign Application Priority Data
Oct. 17, 2014 (JP) 2014-212824

(51) Int. Cl. A61B 17/34; A61B 1/018

(52) U.S. Cl. CPC A61B 17/34; A61B 1/018; A61B 2017/0034

(57) ABSTRACT

A holding mechanism includes an opening portion through which the guide member is inserted in a bending state. The mechanism includes a gap portion which has a cutout surface that is provided by cutting out one part of the holding mechanism in a radial direction of a central axis of an insertion opening portion, and that guides the guide member into the opening portion from the insertion opening portion. The mechanism includes a lock mechanism which is provided in the radial direction and which locks the guide member inserted through the opening portion at two places different from each other in positions in the central axis direction of the insertion opening portion so that one part of the guide member maintains the bending state.

### CROSS-REFERENCE TO RELATED APPLICATIONS

This application is a Continuation Application of PCT Application No. PCT/JP2015/075208, filed Sep. 4, 2015 and based upon and claiming the benefit of priority from prior Japanese Patent Application No. 2014-212824, filed Oct. 17, 2014, the entire contents of all of which are incorporated herein by reference.

### BACKGROUND OF THE INVENTION

1. Field of the Invention

The present invention relates to a holding mechanism which holds a guide member to guide a treatment tool to a subject, and an insertion device having the holding mechanism.

2. Description of the Related Art

For example, Jpn. Pat. Appln. KOKAI Publication No. 2003-116777 discloses a holding member which holds a guide member to guide a treatment tool to a subject. When the holding member holds the guide member, the guide member is positioned and fixed, and the displacement of the guide member is prevented. The holding member is separate from an endoscope, and is fixed to a treatment tool insertion opening portion of the endoscope.

### BRIEF SUMMARY OF THE INVENTION

An aspect according to a holding mechanism of the invention holds a guide member to guide a treatment tool that is inserted into a lumen through an insertion portion of an insertion device to be inserted into the lumen, the holding mechanism includes an opening portion through which the guide member is inserted in a bending state where one part of the guide member exposed to an outside of the insertion device from an insertion opening portion of the insertion device through which the guide member is inserted is bent relative to the insertion opening portion; a gap portion which has a cutout surface that is provided by cutting out one part of the holding mechanism in a radial direction of a central axis of the insertion opening portion, and that guides the guide member into the opening portion from the insertion opening portion; and a lock mechanism which is provided in the radial direction and which locks the guide member inserted through the opening portion at two places different from each other in positions in the central axis direction of the insertion opening portion so that the one part maintains the bending state.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1A is a schematic perspective view of an insertion device according to one embodiment of the present invention;

FIG. 1B is a perspective view of parts around a holding mechanism which is holding a guide member;

FIG. 1C is a perspective view of the parts around the holding mechanism which is not holding the guide member;

FIG. 1D is a side view of the parts around the holding mechanism shown in FIG. 1C;

FIG. 2A is a top view of the parts around the holding mechanism;

FIG. 2B is a diagram in which a first bent pressure-contact part is formed from a state shown in FIG. 2A, and a held part is in contact with a contact portion;

FIG. 2C is a diagram in which the held part has come into contact with an introduction portion from a state shown in FIG. 2B;

FIG. 2D is a diagram in which the held part is locked by a lock mechanism from a state shown in FIG. 2C;

FIG. 3A is a perspective view of the parts around the holding mechanism which is not holding the guide member;

FIG. 3B is a diagram in which the first bent pressure-contact part is formed from a state shown in FIG. 3A, and the held part is in contact with the contact portion;

FIG. 3C is a diagram in which the held part has come into contact with the introduction portion from a state shown in FIG. 3B;

FIG. 3D is a diagram in which the held part has risen from the state shown in FIG. 3B and has been inserted in a lock/introduction portion through an opening portion;

FIG. 3E is a diagram in which the held part has slid on the lock/introduction portion from a state shown in FIG. 3D;

FIG. 3F is a diagram in which the held part has been locked to a first lock portion and a second lock portion from a state shown in FIG. 3E;

FIG. 4 is a cross sectional view of the lock mechanism in a state shown in FIG. 3F;

FIG. 5A shows Modification 1 of the holding mechanism, and is a perspective view of the holding mechanism provided in a forceps plug portion; and

FIG. 5B shows Modification 2 of the holding mechanism, and is a perspective view of parts around a holding mechanism having two holding units.

### DETAILED DESCRIPTION OF THE INVENTION

Hereinafter, an embodiment of the present invention will be described in detail with reference to the drawings.

### One Embodiment

### Configuration

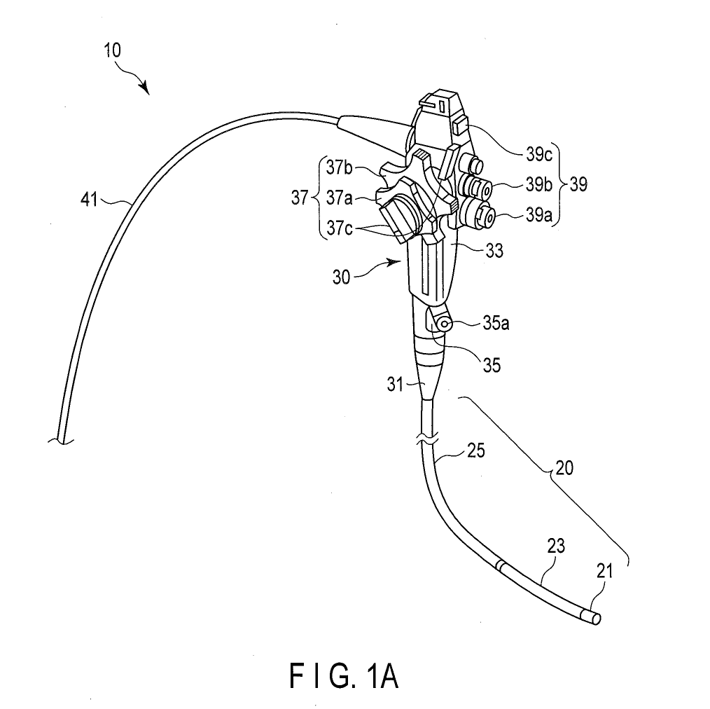

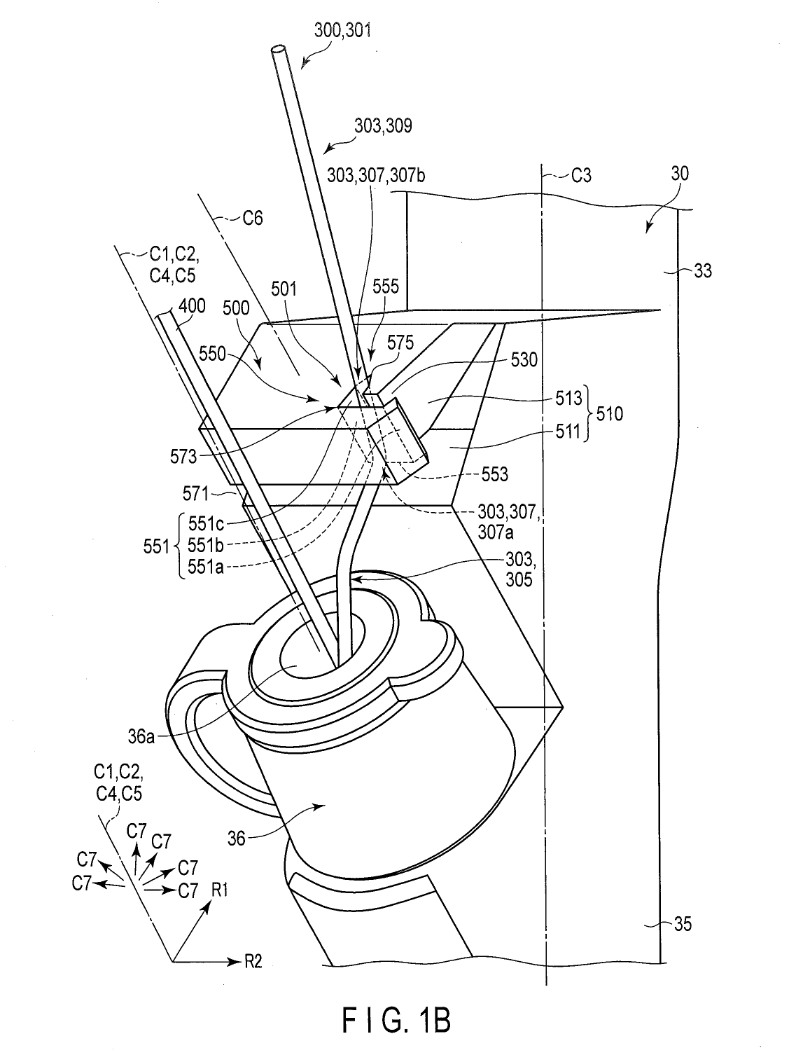

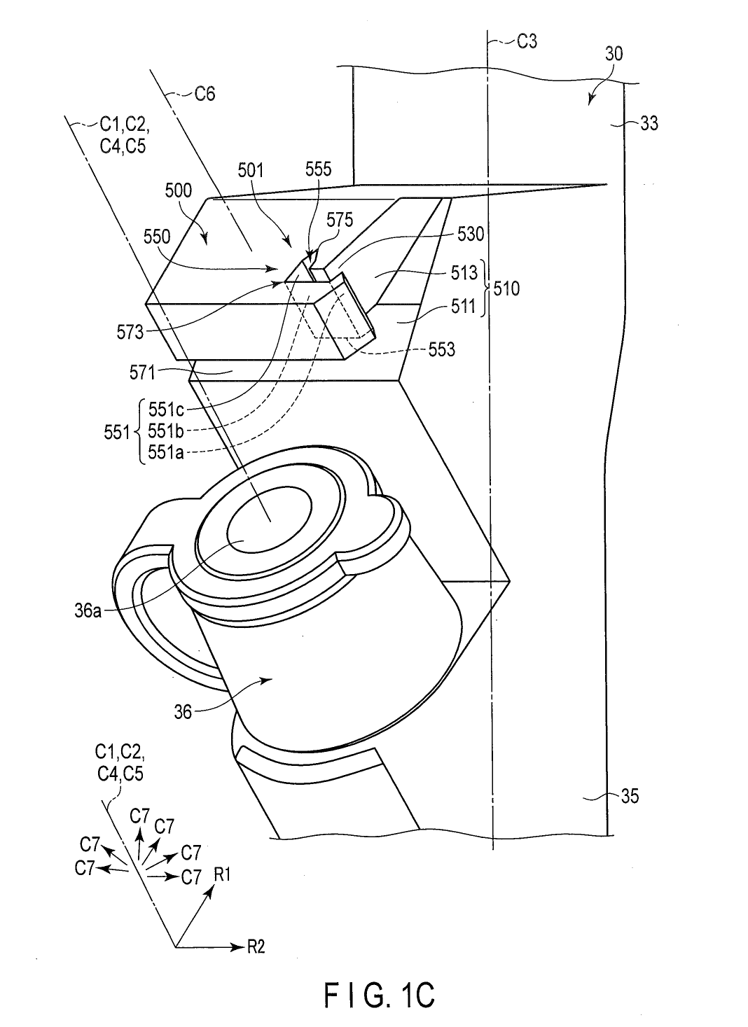

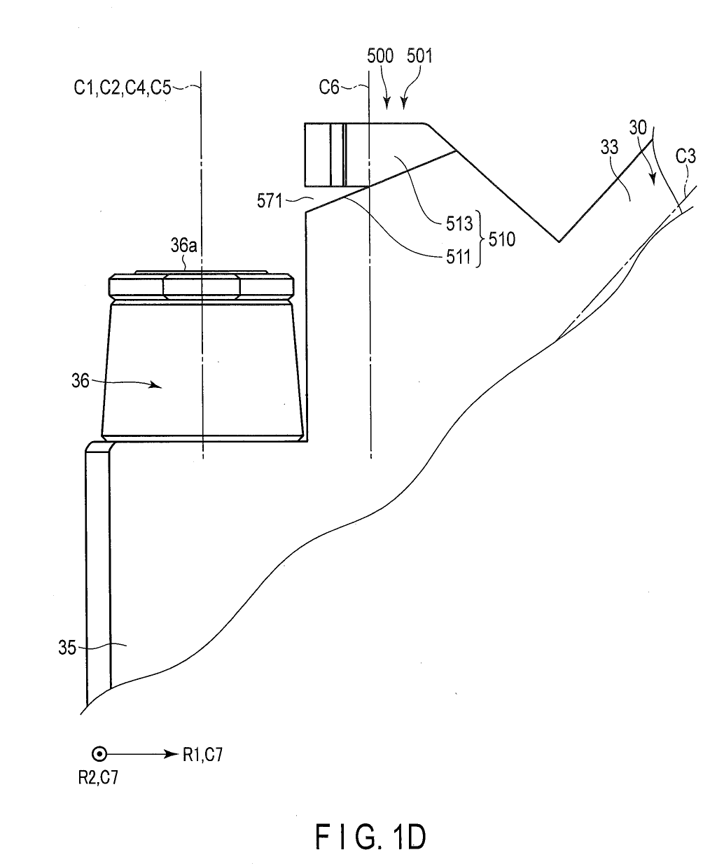

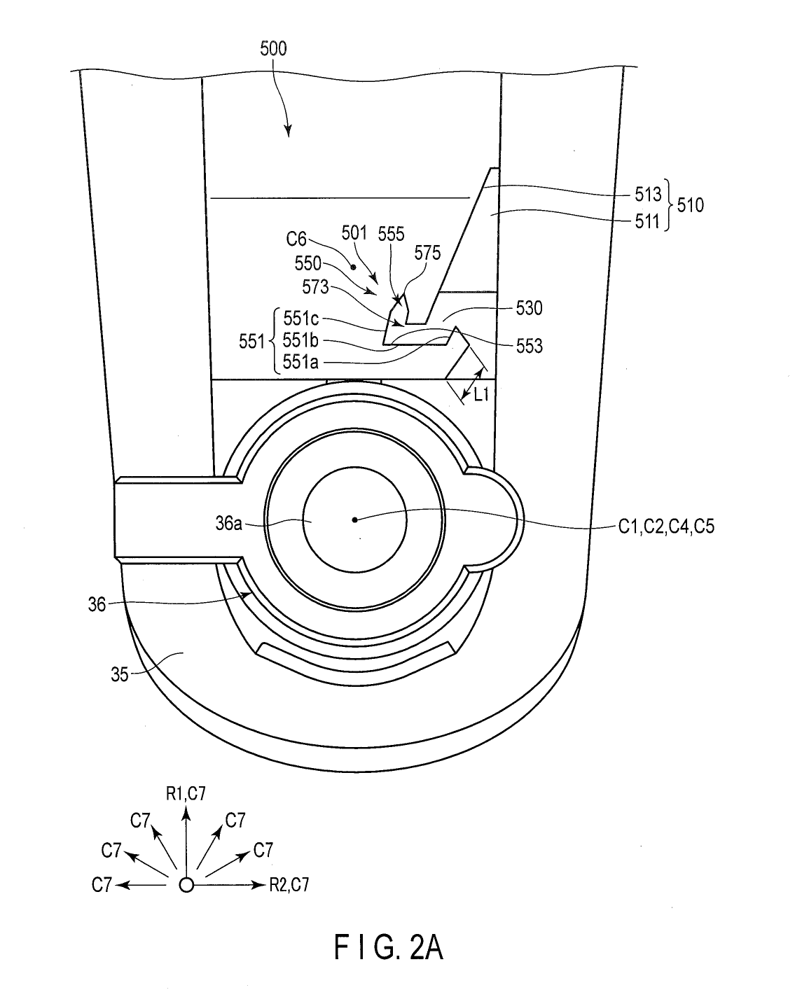

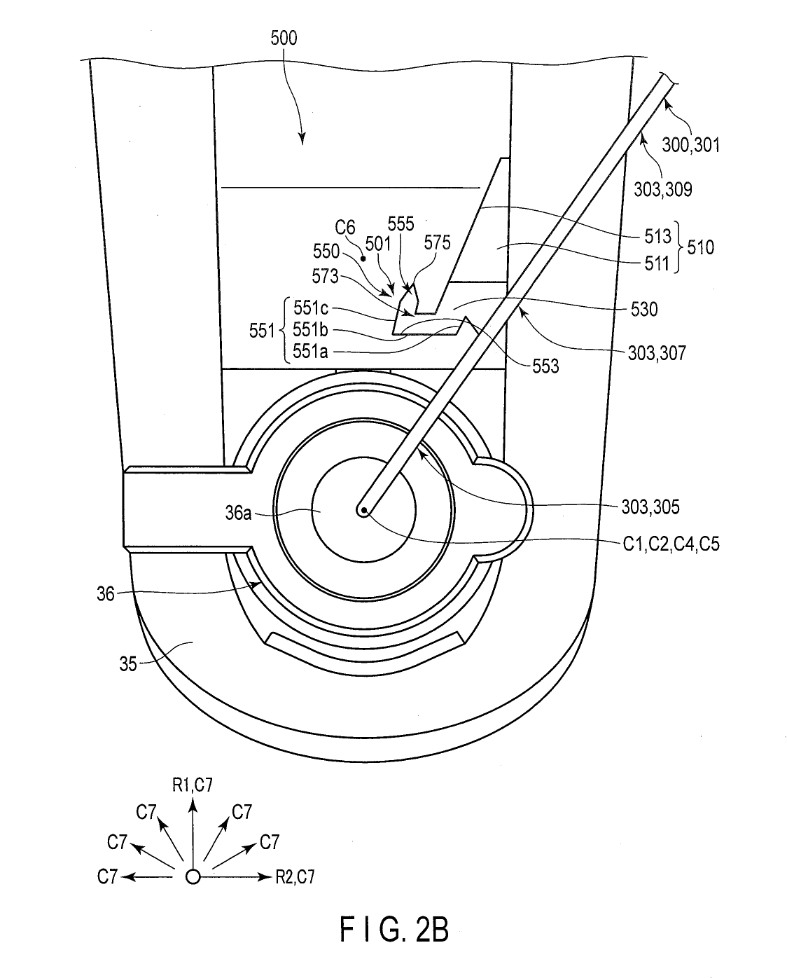

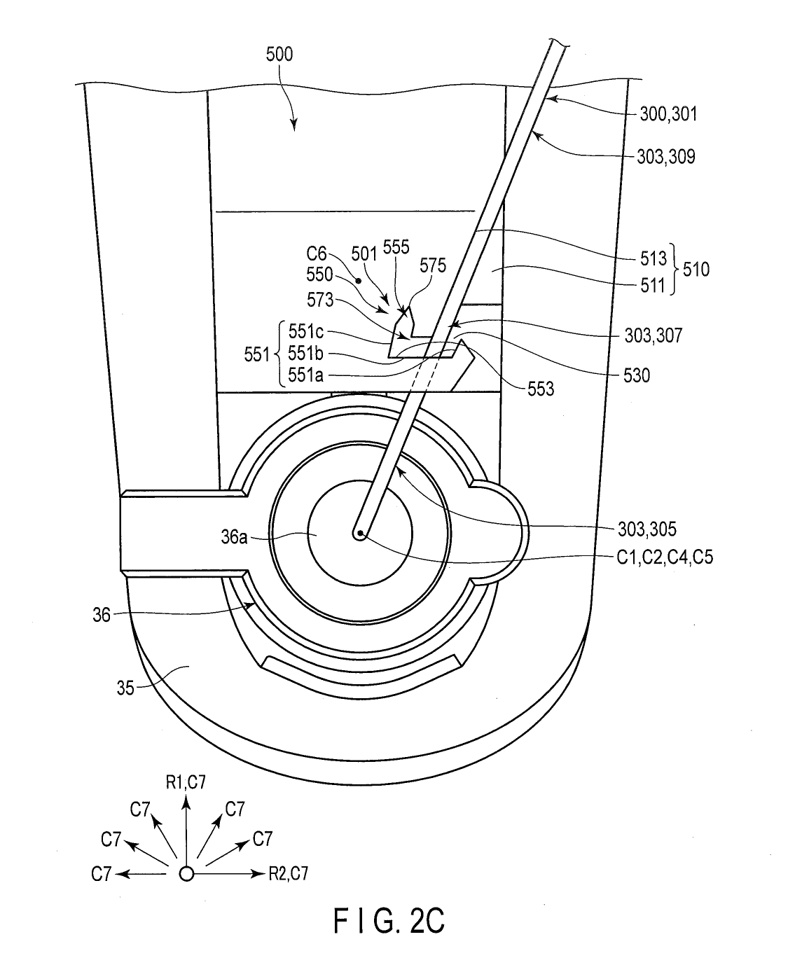

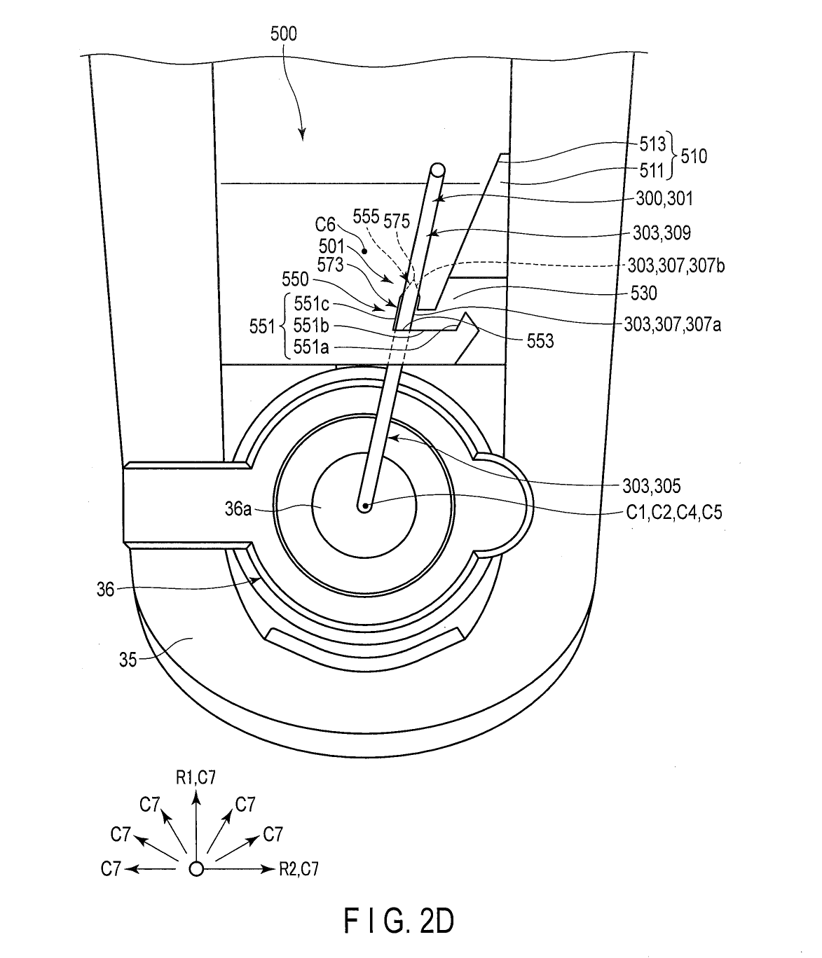

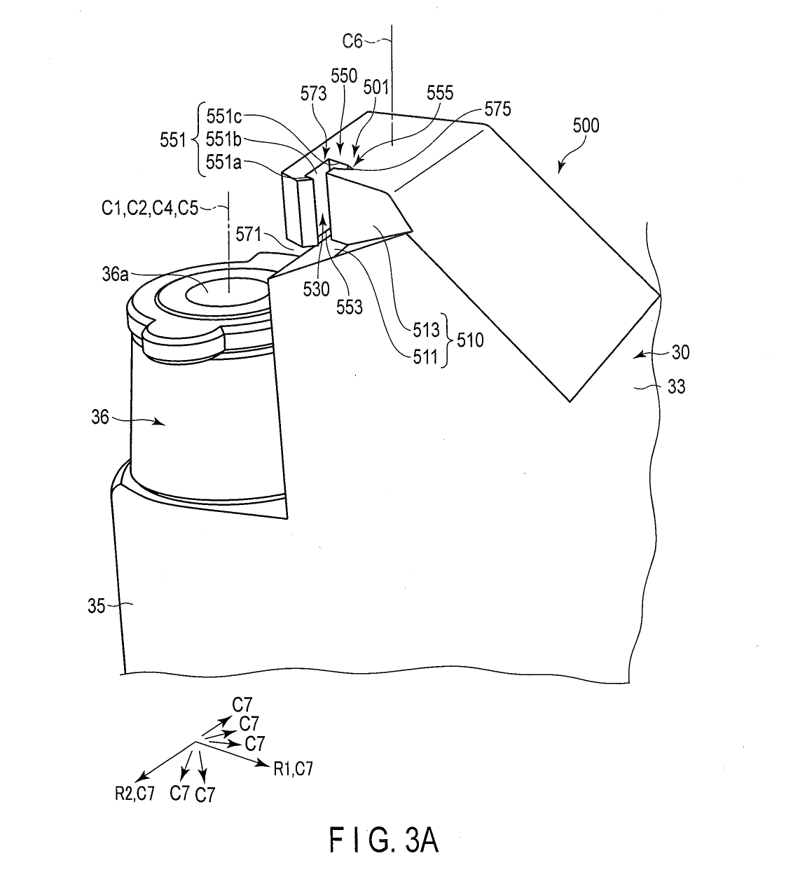

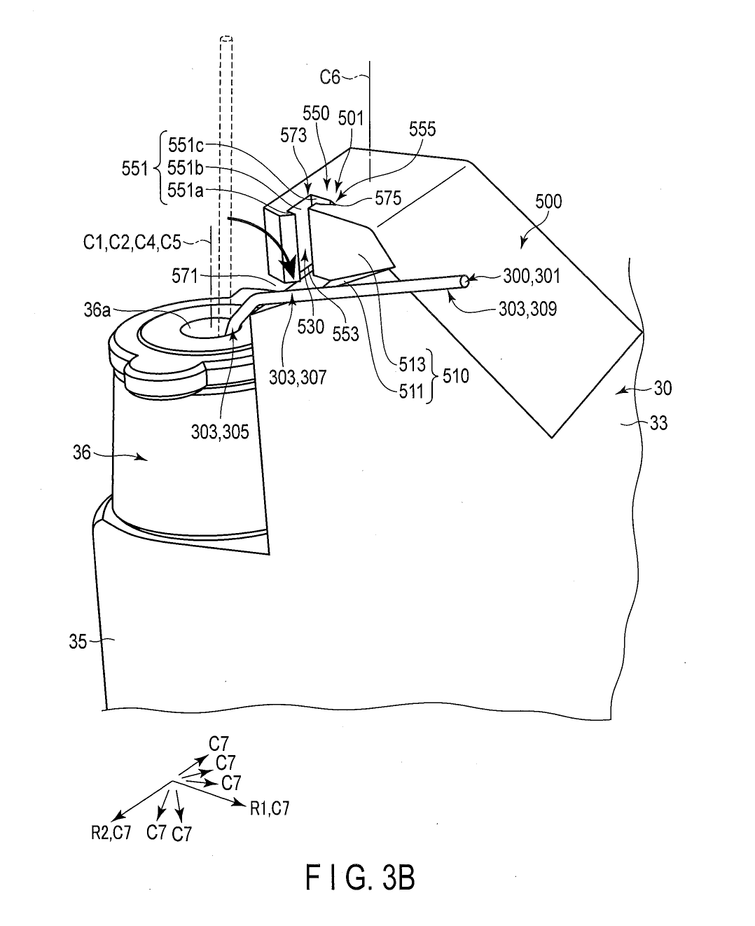

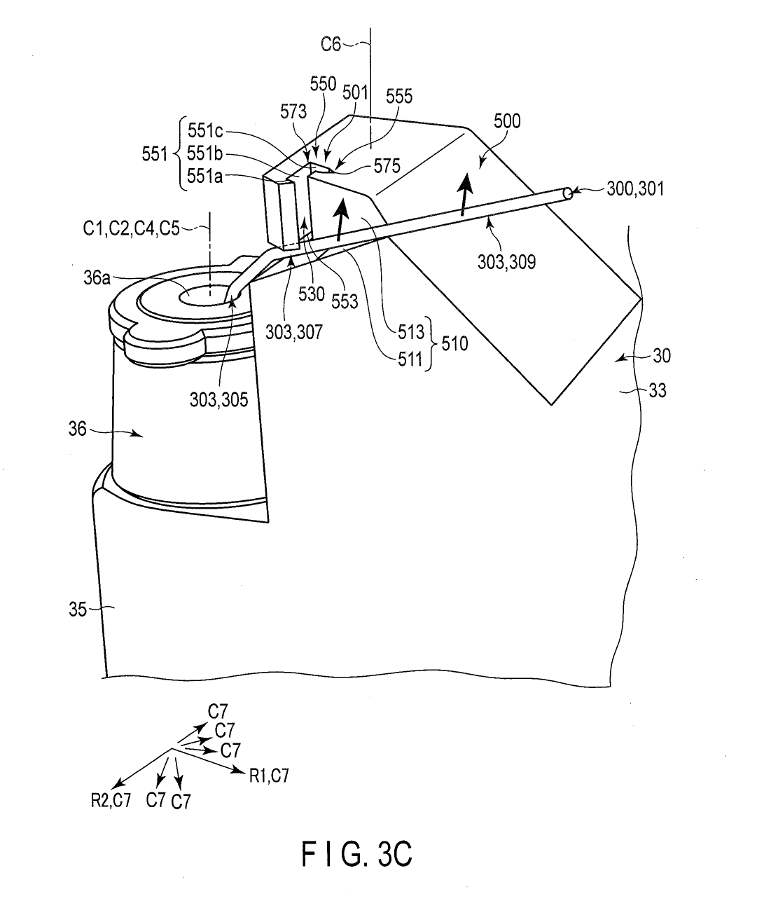

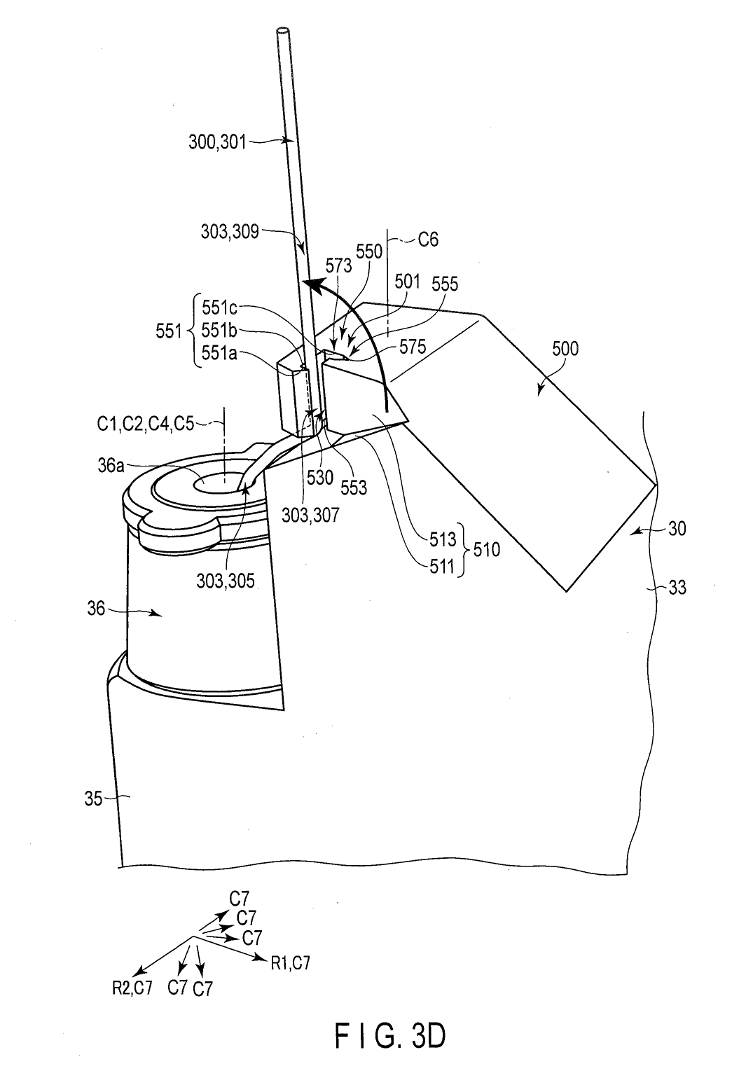

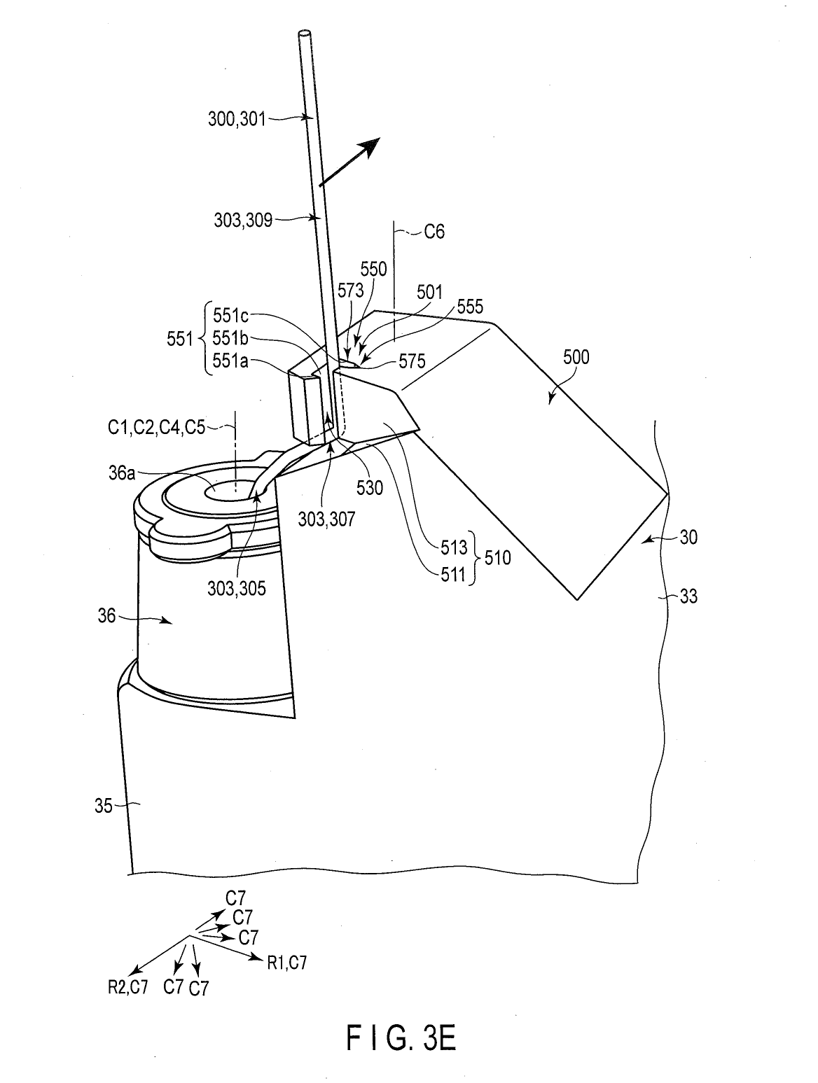

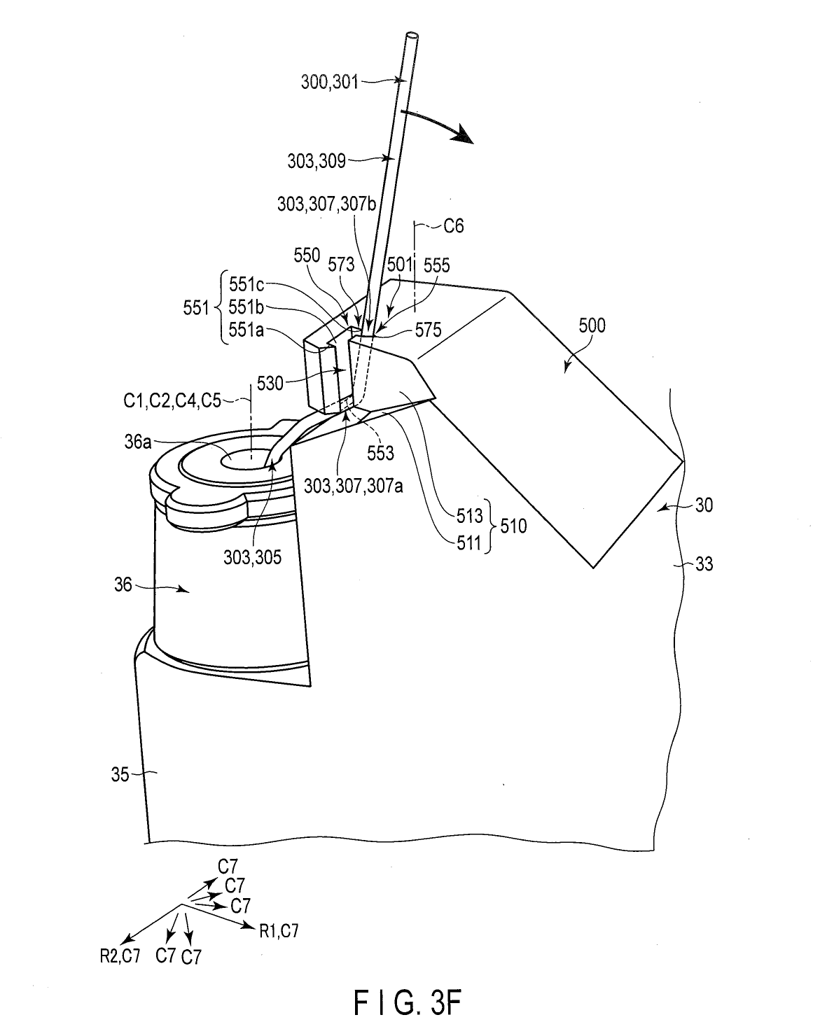

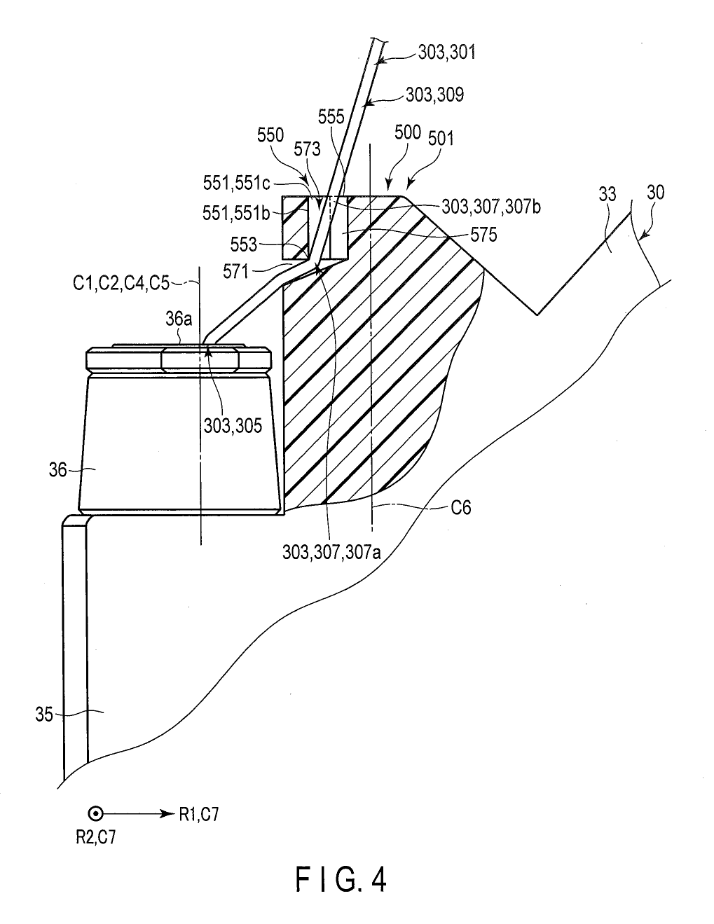

One embodiment is described with reference to FIG. 1A, FIG. 1B, FIG. 1C, FIG. 1D, FIG. 2A, FIG. 2B, FIG. 2C, FIG. 2D, FIG. 3A, FIG. 3B, FIG. 3C, FIG. 3D, FIG. 3E, FIG. 3F, and FIG. 4. For example, as a forceps plug portion 36 and a holding mechanism 500 are omitted in FIG. 1A, some components are omitted for the clarification of the drawings in some of the drawings.

[Insertion Device 10]

An insertion device 10 shown in FIG. 1A functions as, for example, an endoscope to be inserted into a lumen inside a body cavity. The insertion device 10 according to the present embodiment is, for example, a side-view type endoscope which inserts a treatment tool 400 into a duodenal papilla or a biliary and pancreatic area. The insertion device 10 according to the present embodiment may be a direct-view type endoscope, or may be an endoscope which inserts the treatment tool 400 into parts other than the aforementioned parts.

The insertion device 10 according to the present embodiment is described as, for example, a medical endoscope, but does not need to be limited to this. It is also preferable that the insertion device 10 be an industrial endoscope, or an insertion tool such as a catheter having no illumination optical system and no observation optical system, instead of the medical endoscope. This catheter may be a catheter to insert a guide member into the lumen.

As shown in FIG. 1A, the insertion device 10 has a hollow and elongated insertion portion 20 to be inserted into the lumen, and an operation portion 30 which is coupled to a proximal end portion of the insertion portion 20 and which operates the insertion device 10.

[Insertion Portion 20]

As shown in FIG. 1A, the insertion portion 20 has a distal hard portion 21, a bendable portion 23, and a flexible tube portion 25 from a distal end portion side of the insertion portion 20 toward the proximal end portion side of the insertion portion 20. A proximal end portion of the distal hard portion 21 is coupled to a distal end portion of the bendable portion 23, and a proximal end portion of the bendable portion 23 is coupled to a distal end portion of the flexible tube portion 25.

The distal hard portion 21 is the distal end portion of the insertion portion 20, and is hard and non-bendable. When the insertion device 10 is the side-view type endoscope, the distal hard portion 21 has an unshown existing configuration. That is, the distal hard portion 21 has an unshown distal opening portion provided in a side surface of the distal hard portion 21, and an unshown treatment tool raising support which is housed in the distal opening portion and which can be remotely operated to swing in response to a raising operation at hand. The treatment tool raising support swings between a housing position at which the treatment tool raising support is collapsed relative to a body portion of the distal hard portion 21 and housed in the distal hard portion 21 and a raise position at which the treatment tool raising support protrudes (rises) from the distal opening portion. The treatment tool raising support swings in response to the raising operation, and guides and protrudes the treatment tool 400 to the lateral side of the distal hard portion 21 from the distal opening portion. The distal hard portion has an air/water nozzle, an imaging unit, and an illumination unit.

The bendable portion 23 bends in a desired direction such as an upward, downward, rightward, and leftward direction in accordance with the operation of a later-described bending operation portion 37. The bendable portion 23 bends so that the position and direction of the distal hard portion 21 change. Unshown illumination light is applied to an observation target, and the observation target is caught in an observation field of view. This observation target is, for example, an affected part and a lesion in a subject (e.g. the body cavity).

The flexible tube portion 25 has desired flexibility. Therefore, the flexible tube portion 25 flexes in response to an external force. The flexible tube portion 25 is a tubular member extending from a later-described body portion 31 in the operation portion 30.

[Operation Portion 30]

As shown in FIG. 1A, the operation portion 30 has the body portion 31 in which the flexible tube portion 25 extends, a grasp portion 33 which is coupled to a proximal end portion of the body portion 31 and which is grasped by an operator who operates the insertion device 10, and a universal cord 41 connected to the grasp portion 33.

[Grasp Portion 33]

As shown in FIG. 1A, the grasp portion 33 has a treatment tool insertion portion 35, the bending operation portion 37 which is operated to bend the bendable portion 23, and a switch portion 39. The treatment tool insertion portion 35 is provided on a distal end portion side of the grasp portion 33, and the bending operation portion 37 and the switch portion 39 are provided on a proximal end portion side of the grasp portion 33.

[Treatment Tool Insertion Portion 35]

As shown in FIG. 1A, the treatment tool insertion portion 35 branches relative to the grasp portion 33. Thus, a central axis C2 direction of the treatment tool insertion portion 35 is inclined relative to a central axis C3 direction of the grasp portion 33.

As shown in FIG. 1A, the treatment tool insertion portion 35 has a treatment tool insertion opening portion 35a which is provided at an end portion of the treatment tool insertion portion 35 and which is used to insert a later-described guide member 300 and the treatment tool 400 into the insertion device 10.

The treatment tool insertion opening portion 35a is coupled to a proximal end portion of an unshown treatment tool insertion channel. The treatment tool insertion channel is provided inside the insertion portion 20, and provided to extend to the distal hard portion 21 from the flexible tube portion 25 via the bendable portion 23. A distal end portion of the treatment tool insertion channel communicates with the distal opening portion provided in the distal hard portion 21. The treatment tool insertion opening portion 35a is an insertion opening portion to insert the guide member 300 and the treatment tool 400 into the treatment tool insertion channel.

As shown in FIG. 1A, a central axis C1 of the treatment tool insertion opening portion 35a is provided coaxially with the central axis C2 of the treatment tool insertion portion 35, and is therefore inclined relative to the central axis C3 of the grasp portion 33. The central axis C1 direction of the treatment tool insertion opening portion 35a is inclined relative to the central axis direction C3 of the grasp portion 33.

As shown in FIG. 1B, the treatment tool insertion portion 35 has the cylindrical forceps plug portion 36 detachably provided in the treatment tool insertion portion 35. The forceps plug portion 36 is made of a resin such as rubber. A central axis C4 of the forceps plug portion 36 is provided coaxially with the central axis C1 of the treatment tool insertion opening portion 35a. Therefore, the forceps plug portion 36 is inclined relative to the grasp portion 33. When the forceps plug portion 36 is provided in the treatment tool insertion portion 35, the forceps plug portion 36 communicates with the treatment tool insertion channel via the treatment tool insertion opening portion 35a. The cylindrical forceps plug portion 36 has an insertion opening portion 36a to insert the guide member 300 and the treatment tool 400 into the insertion device 10, specifically, into the treatment tool insertion channel. A central axis C5 of the insertion opening portion 36a is provided coaxially with the central axis C4 of the forceps plug portion 36.

The guide member 300 and the treatment tool 400 shown in FIG. 1B are inserted into the treatment tool insertion channel from the insertion opening portion 36a via the forceps plug portion 36 and the treatment tool insertion opening portion 35a, and pressed up to the distal hard portion 21 side. The guide member 300 and the treatment tool 400 protrude from the distal opening portion.

[Bending Operation Portion 37]

As shown in FIG. 1A, the bending operation portion 37 has a right/left operation knob 37a which is operated to bend the bendable portion 23 rightward and leftward, an up/down operation knob 37b which is operated to bend the bendable portion 23 upward and downward, and a fixing knob 37c which fixes the position of the bent bendable portion 23.

[Switch Portion 39]

As shown in FIG. 1A, the switch portion 39 has a suction switch 39a, an air/water switch 39b, and a variety switch 39c for endoscopic photography. The suction switch 39a, the air/water switch 39b, and the variety switch 39c are operated by the hand of the operator when the grasp portion 33 is grasped by the operator.

The suction switch 39a is operated when the insertion device 10 sucks a fluid including mucus from the aforementioned distal opening portion which functions as a suction opening portion via the treatment tool insertion channel which functions as a suction channel.

The air/water switch 39b is operated when the fluid is supplied from an unshown air supply tube and an unshown air/water supply tube and when the fluid is supplied from a water supply tube and the air/water supply tube to ensure the observation field of view of the unshown imaging unit in the distal hard portion 21. The fluid includes water and gasses.

The air supply tube, the water supply tube, and the air/water supply tube are provided up to the universal cord 41 from the insertion portion 20 via the body portion 31 and the grasp portion 33 inside the insertion device 10.

[Universal Cord 41]

As shown in FIG. 1A, the universal cord 41 extends from the side surface of the grasp portion 33.

[Guide Member 300, Treatment Tool 400, and Holding Mechanism 500]

As shown in FIG. 1E, the insertion device 10 is used together with the guide member 300 and the treatment tool 400 which are separate from the insertion device 10. Thus, the insertion device 10 has the holding mechanism 500 which holds the guide member 300. The guide member 300 is inserted into the treatment tool insertion channel from the insertion opening portion 36a via the forceps plug portion 36, protrudes from the distal opening portion and reaches the subject, and guides the treatment tool 400 up to the subject. Thus, the holding mechanism 500 holds the guide member 300 to guide the treatment tool 400 which is inserted into the lumen through the insertion portion 20 of the insertion device 10 to be inserted into the lumen. The guide member 300 is disposed so that the guide member 300 is movable in an axial direction of the guide member 300 in a state where the guide member 300 is protruding from the distal opening portion provided at the distal end of the insertion portion 20. The guide member 300 guides the treatment tool 400 by being disposed as above.

[Guide Member 300]

As shown in FIG. 1B, the guide member 300 is inserted into the treatment tool insertion channel from the insertion opening portion 36a via the forceps plug portion 36 and the treatment tool insertion opening portion 35a. A distal end portion of the guide member 300 is then inserted through the treatment tool insertion channel, and protrudes from the distal opening portion. The distal opening portion is provided in the side surface, and the distal end portion of the guide member 300 therefore protrudes toward the side surface of the insertion portion 20. The distal end portion of the guide member 300 then reaches the subject. In a state where the distal end portion of the guide member 300 is protruding to an outside the insertion device 10 from the distal opening portion, a proximal end portion 301 of the guide member 300 is exposed to the outside of the insertion device 10 from the insertion opening portion 36a.

As shown in FIG. 1B, at the exposed proximal end portion 301, a part 303 of the guide member 300 immediately after exposed to the outside from the insertion opening portion 36a is bent, for example, in a substantially S-shape, for example, toward the grasp portion 33 of the operation portion 30 from the insertion opening portion 36a. The part 303 is brought into pressure contact with the forceps plug portion 36 and the holding mechanism 500 in a bending state. This will be described below.

As shown in FIG. 1B, at the exposed proximal end portion 301, the part 303 of the guide member 300 immediately after exposed to the outside from the insertion opening portion 36a is in a bending state to be bent, for example, toward the grasp portion 33 of the operation portion 30 from the insertion opening portion 36a and in a pressure contact state to be in pressure contact with the forceps plug portion 36 including the insertion opening portion 36a.

This part is referred to as a first bent pressure-contact part 305 for convenience of explanation. Details of the bending state and the pressure contact state will be described later.

Another part 303 is continuous with the first bent pressure-contact part 305, is adjacent to the first bent pressure-contact part 305, and is held by the holding mechanism 500.

This part is referred to as a held part 307 for convenience of explanation.

The held part 307 has a second bent pressure-contact part 307a and a pressure contact part 307b. The second bent pressure-contact part 307a is a part which is bent and in pressure contact with the holding mechanism 500 by being bent. The pressure contact part 307b is a part which is in pressure contact with the holding mechanism 500 when the bent second bent pressure-contact part 307a will be restored to a straight shape by a restoring force of the guide member 300. The restoring force includes the resiliency of the guide member 300. The second bent pressure-contact part 307a and the pressure contact part 307b are provided inside the holding mechanism 500.

The second bent pressure-contact part 307a is provided closer to the proximal side of the guide member 300 with respect to the first bent pressure-contact part 305. The second bent pressure-contact part 307a is linearly continuous with the first bent pressure-contact part 305. The pressure contact part 307b is provided closer to the proximal side of the guide member 300 with respect to the second bent pressure-contact part 307a. The pressure contact part 307b is linearly continuous with the second bent pressure-contact part 307a.

Another part 303 is continuous with the held part 307, is adjacent to the held part 307, and extends straight, for example, toward a front of the grasp portion 33 of the operation portion 30.

This part is referred to as an extending part 309 for convenience of explanation. The extending part 309 is a part which is not held by the holding mechanism 500 and which extends toward an outside of the holding mechanism 500 from the holding mechanism 500. The extending part 309 is formed straight by the resiliency of the guide member 300. The extending part 309 is located within an angle formed between the central axis C3 of the grasp portion 33 and the central axis C1 of the treatment tool insertion opening portion 35a.

As shown in FIG. 1B, the proximal end portion 301 which is exposed as above has the first bent pressure-contact part 305, the held part 307, and the extending part 309. At the proximal end portion 301, the first bent pressure-contact part 305, the held part 307, and the extending part 309 are arranged in this order from the distal end portion of the guide member 300 toward the proximal end portion 301. As described above, the first bent pressure-contact part 305, the held part 307, and the extending part 309 are parts separate from one another. The extending part 309 is longer than the first bent pressure-contact part 305 and the held part 307. The held part 307 and the extending part 309 are provided closer to the grasp portion 33 side with respect to the first bent pressure-contact part 305.

The guide member 300 is inserted into the treatment tool insertion channel from the insertion opening portion 36a via the forceps plug portion 36 simultaneously with or before the treatment tool 400 depending on operative methods. The guide member 300 then guides the treatment tool 400 to the subject.

Such a guide member 300 is made of, for example, a thin linear material. The linear material has, for example, a hard metal wire having strong resiliency. The resiliency inhibits the effect of a bending tendency on the guide member 300. The bending tendency indicates that the guide member 300 maintains the bending state when the guide member 300 has bent. The guide member 300 will be restored to, for example, a straight state due to the resiliency even if bent. The aforementioned metal includes, for example, at least one of a nickel-titanium alloy and stainless steel. The linear material may have, for example, a nickel-titanium wire, and, for example, a fluorine resin with which the surface of the nickel-titanium wire is coated. The guide member 300 is, for example, disposable.

[Treatment Tool 400]

The treatment tool 400 shown in FIG. 1B is used to treat the subject such as a bile duct. Such a treatment tool 400 has, for example, a knife for endoscopic sphincterotomy (hereinafter, EST). The treatment tool 400 is made of, for example, a thin linear material.

The treatment tool 400 is guided to the subject by the guide member 300 by desired timing suited to an operative method.

[Holding Mechanism 500]

The holding mechanism 500 is provided in the operation portion 30 so that the holding mechanism 500 can hold or unhold the held part 307 by an operation with a left hand of a surgeon in a state where the grasp portion 33 is grasped with a right hand of the surgeon. In other words, the holding mechanism 500 is provided in the operation portion 30 so that the held part 307 is attachable to and detachable from the grasp portion 33 via the holding mechanism 500 by the operation with the left hand of the surgeon in a state where the grasp portion 33 is grasped with the right hand of the surgeon. Thus, the holding mechanism 500 is provided in the operation portion 30 so that the holding mechanism 500 may not interfere with the operation by the right hand of the surgeon grasping the insertion device 10 and so that the guide member 300, the treatment tool 400, and the holding mechanism 500 may be operable by the operation with the left hand.

In general, the treatment tool 400 moves back and forth along the axial direction of the treatment tool 400 by the operation with the right hand of the surgeon in a state where the grasp portion 33 is grasped with the left hand of the surgeon. In the present embodiment, the held part 307 is attached to the grasp portion 33 via the holding mechanism 500 by the operation with the left hand of the surgeon in a state where the grasp portion 33 is grasped with the right hand of the surgeon. The surgeon then transfers the grasp portion 33 from the right hand to the left hand and grasps the grasp portion 33 with the left hand.

As shown in FIG. 1B, FIG. 1C, FIG. 1D, and FIG. 3A, the holding mechanism 500 is provided in the operation portion 30 which is coupled to the proximal end portion of the insertion portion 20. In this case, the holding mechanism 500 is provided adjacent to the insertion opening portion 36a, specifically, the forceps plug portion 36 as shown in FIG. 1B. The holding mechanism 500 is provided between the forceps plug portion 36 and the grasp portion 33. A central axis C6 of the holding mechanism 500 is offset relative to the central axis C5 of the insertion opening portion 36a (the forceps plug portion 36) so that the central axis C6 of the holding mechanism 500 is provided between the central axis C3 of the grasp portion 33 and the central axis C5 of the insertion opening portion 36a (the forceps plug portion 36). In other words, the central axis C6 of the holding mechanism 500 is substantially parallel to the central axis C5 of the insertion opening portion 36a.

As shown in FIG. 1B, FIG. 1C, FIG. 1D, and FIG. 3A, the holding mechanism 500 is integral with, for example, the grasp portion 33 and the treatment tool insertion portion 35 of the operation portion 30, or is a separate body attachable to and detachable from the operation portion 30, which is, however, not shown.

As shown in FIG. 1B, FIG. 1C, FIG. 1D, and FIG. 3A, when the holding mechanism 500 is integral with the operation portion 30, for example, the holding mechanism 500 is integral with the side surface of the grasp portion 33 and a flat surface of the treatment tool insertion portion 35 in which the forceps plug portion 36 is provided. Such a holding mechanism 500 is insert-molded together with the grasp portion 33.

Although not shown, when the holding mechanism 500 is separate from the operation portion 30, the holding mechanism 500 is detachably attached to, for example, the grasp portion 33 and the treatment tool insertion portion 35. In this case, the holding mechanism 500 may be attached to the grasp portion 33 and the treatment tool insertion portion 35 by adhesive bonding.

As shown in FIG. 1B, FIG. 1C, FIG. 1D, and FIG. 3A, the holding mechanism 500 has one holding unit 501. The holding unit 501 is provided more to the right with respect to the central axis C3 of the grasp portion 33 when the holding mechanism 500 is seen from the forceps plug portion 36 toward the grasp portion 33.

As shown in FIG. 1B, FIG. 1C, FIG. 1D, FIG. 2A, and FIG. 3A, the holding unit 501 has a guide portion 510 having a contact portion 511 and an introduction portion 513, an opening portion 530, a lock mechanism 550, and a gap portion 571. The guide portion 510, the opening portion 530, the lock mechanism 550, and the gap portion 571 are formed by cutting out a thick portion of the holding mechanism 500. The guide portion 510 is continuous with the lock mechanism 550, and the opening portion 530 is provided between the guide portion 510 and the lock mechanism 550. The contact portion 511, the introduction portion 513, and the lock mechanism 550 are continuous peripheral surfaces of the holding mechanism 500, and the guide member 300 slides on these parts. In other words, the guide member 300 moves on these parts along these parts. In this state, the order in the path to hold the guide member 300 is the gap portion 571, the contact portion 511, the introduction portion 513, the opening portion 530, and the lock mechanism 550. Each part is described below.

As shown in FIG. 2A, FIG. 2B, FIG. 3A, and FIG. 3B, the guide portion 510 is provided so that in the bending state where one part 303 (the first bent pressure-contact part 305) of the guide member 300 immediately after exposed to the outside from the insertion opening portion 36a is bent relative to the insertion opening portion 36a and in the pressure contact state where one part 303 (the first bent pressure-contact part 305) is in pressure contact with the forceps plug portion 36 including the insertion opening portion 36a, another exposed part 303 (the held part 307 and a part of the extending part 309) contacts the guide portion 510. In other words, the guide portion 510 is provided so that the held part 307 and the one part of the extending part 309 contact the guide portion 510 in a state where the first bent pressure-contact part 305 is formed. The guide portion 510 guides the held part 307 and the part of the extending part 309 that are in contact with the guide portion 510 to a lock/introduction portion 551 of the lock mechanism 550. The guide portion 510 functions as a smooth slide surface on which the proximal end portion 301 of the guide member 300 including the held part 307 and the part of the extending part 309 slide. The guide portion 510 is provided, for example, between the forceps plug portion 36 and the grasp portion 33 in a first diametrical direction R1 of the insertion opening portion 36a (the length direction of the holding mechanism 500).

As shown in FIG. 1B, FIG. 2E, and FIG. 3B, the bending state indicates that, for example, the first bent pressure-contact part 305 of the part 303 bends in, for example, a arc-shape from the central axis C5 direction of the insertion opening portion 36a (the forceps plug portion 36) in a substantially orthogonal direction that intersects substantially at right angles with respect to the central axis C5 direction, and the first bent pressure-contact part 305 of the part 303 further bends toward, for example, the grasp portion 33 of the operation portion 30 from the insertion opening portion 36a (the forceps plug portion 36).

As shown in FIG. 1B, FIG. 2B, and FIG. 3B, the pressure contact state indicates that the bent part 303 (the first bent pressure-contact part 305) is in pressure contact with an inner circumferential surface and an upper-edge-side inner circumferential edge of the insertion opening portion 36a (the forceps plug portion 36) from the insertion opening portion 36a (the forceps plug portion 36) toward, for example, the grasp portion 33 of the operation portion 30, and the bent part 303 is further in pressure contact with an upper end surface of the forceps plug portion 36 from the upper side than the insertion opening portion 36a (the forceps plug portion 36) toward the upper end surface of the forceps plug portion 36 that is a flat surface in which the insertion opening portion 36a is provided, and is positioned and fixed by the pressure contact.

As shown in FIG. 1B, FIG. 1C, FIG. 2B, FIG. 2C, FIG. 3B, and FIG. 3C, the guide portion 510 has the contact portion 511 with which the held part 307 and the part of the extending part 309 contact, and the introduction portion 513 which intervenes between the contact portion 511 and the lock mechanism 550 in a guide direction and which is continuous with the contact portion 511 and the lock mechanism 550. The guide portion 510 guides the held part 307 and the part of the extending part 309 toward the lock mechanism 550 from the contact portion 511 via the introduction portion 513.

As shown in FIG. 1B and FIG. 1C, the contact portion 511 is formed by cutting out the thick portion of the holding mechanism 500 from the forceps plug portion 36 toward the grasp portion 33 in the first diametrical direction R1 of the insertion opening portion 36a. Thus, the gap portion 571 having a cutout surface is formed between the lock mechanism 550 and the contact portion 511 in the central axis C5 direction of the insertion opening portion 36a. The cutout surface is provided by cutting out a part of the holding mechanism 500 in a radial direction C7 of the central axis C5 of the insertion opening portion 36a, and guides the guide member 300 into the opening portion 530 from the insertion opening portion 36a. The cutout surface has, for example, the contact portion 511 and the introduction portion 513. The radial direction C7 is, for example, a planar direction including the first diametrical direction R1 and a second diametrical direction R2 of the insertion opening portion 36a.

The contact portion 511 pierces the thick portion of the holding mechanism 500 in the second diametrical direction R2 of the insertion opening portion 36a (the width direction of the holding mechanism 500) that intersects at right angles with the first diametrical direction R1. The contact portion 511 has only to be provided more to the right with respect to the central axis C3 of the grasp portion 33 and the central axis C4 of the forceps plug portion 36 when the holding mechanism 500 is seen from the forceps plug portion 36 toward the grasp portion 33. The contact portion 511 is exposed to the outside via the right side, upper side, and near side of the holding mechanism 500 when the holding mechanism 500 is seen from the forceps plug portion 36 toward the grasp portion 33.

As shown in FIG. 1B, FIG. 1C, and FIG. 1D, the contact portion 511 is a flat surface formed between the first diametrical direction R1 and the second diametrical direction R2 of the insertion opening portion 36a. The contact portion 511 functions as a bottom surface of the guide portion 510. A lowest part of the contact portion 511 is provided at a position higher than the upper end portion of the forceps plug portion 36. The lowest part of the contact portion 511 is adjacent to the forceps plug portion 36 in the first diametrical direction R1 of the insertion opening portion 36a. The contact portion 511 is inclined relative to the upper end face of the forceps plug portion 36. The contact portion 511 is bent toward the grasp portion 33 from the forceps plug portion 36 in the first diametrical direction R1 of the insertion opening portion 36a. The whole contact portion 511 is formed as an inclined surface. In the first diametrical direction R1 of the insertion opening portion 36a, the forceps plug portion 36, the lowest part of the contact portion 511, the contact portion 511, the highest part of the contact portion 511, and the grasp portion 33 are arranged in this order.

As shown in FIG. 1B and FIG. 1C, the introduction portion 513 is a flat surface formed between the first diametrical direction R1 of the insertion opening portion 36a and the central axis C5 of the insertion opening portion 36a. The introduction portion 513 functions as a right side surface of the holding mechanism 500. The introduction portion 513 is provided between the forceps plug portion 36 and the grasp portion 33 in the first diametrical direction R1 of the insertion opening portion 36a. The introduction portion 513 is linearly provided in the first diametrical direction R1 of the insertion opening portion 36a. A inclination of the introduction portion 513 (the direction of the introduction portion 513) relative to a straight line that connects the central axis C5 of the insertion opening portion 36a to the central axis C3 of the grasp portion 33 is adjusted as desired. Thus, the introduction portion 513 is inclined relative to the central axis C5 of the insertion opening portion 36a.

As shown in FIG. 2C, FIG. 3C, and FIG. 3D, the introduction portion 513 introduces the held part 307 and the part of the extending part 309 from the contact portion 511 toward the lock mechanism 550. In this instance, the introduction portion 513 functions as a raising slide surface on which the held part 307 and the part of the extending part 309 slide so that the held part 307 and the part of the extending part 309 that are provided along the first diametrical direction R1 of the insertion opening portion 36a rise along the central axis C5 direction of the insertion opening portion 36a.

As shown in FIG. 1B and FIG. 1C, the opening portion 530 and the lock mechanism 550 are provided more to the near side (the forceps plug portion 36) with respect to the introduction portion 513. In other words, the opening portion 530 and the lock mechanism 550 are provided between the introduction portion 513 and the forceps plug portion 36 in the first diametrical direction R1 of the insertion opening portion 36a. The opening portion 530 and the lock mechanism 550 are formed as a J-shaped groove portion. Thus, in the first diametrical direction R1 of the insertion opening portion 36a, the thick portion of the holding mechanism 500 is provided between the opening portion 530 and the lock mechanism 550, and the forceps plug portion 36.

As shown in FIG. 1B and FIG. 1C, the opening portion 530 and the lock mechanism 550 are provided along the central axis C5 direction of the insertion opening portion 36a, and pierce the thick portion of the holding mechanism 500 in the central axis C5 direction of the insertion opening portion 36a. Thus, a part of the contact portion 511 is provided under the opening portion 530 and the lock mechanism 550.

As shown in FIG. 3C and FIG. 3D, the opening portion 530 functions as a communication path in which the held part 307 is provided to be inserted into or removed from the lock mechanism 550. The part 303 is inserted through the opening portion 530 in the bending state where the first bent pressure-contact part 305 of the part 303 on the proximal side of the guide member 300 immediately after exposed to the outside from the insertion opening portion 36a is bent relative to the insertion opening portion 36a and in the pressure contact state where the first bent pressure-contact part 305 is in pressure contact with the insertion opening portion 36a.

As shown in FIG. 1B, FIG. 2D, and FIG. 3D, the lock mechanism 550 is continuous with the opening portion 530. The held part 307 which has been inserted through the opening portion 530 is inserted into the lock mechanism 550. The lock mechanism 550 is provided in the radial direction C7 of the central axis C5 of the insertion opening portion 36a. Specifically, the lock mechanism 550 is provided at a position farther from the insertion opening portion 36a in the central axis C5 direction of the insertion opening portion 36a. At a position higher than the insertion opening portion 36a, the lock mechanism 550 is provided adjacent to the insertion opening portion 36a and on the radial direction C7 of the central axis C5 of the insertion opening portion 36a. The central axis of the lock mechanism 550 is parallel to the central axis C5 of the insertion opening portion 36a. When the holding mechanism 500 is seen from the forceps plug portion 36 toward the grasp portion 33, the lock mechanism 550 is provided between the forceps plug portion 36 and the grasp portion 33.

As shown in FIG. 1B, FIG. 2D, FIG. 3F, and FIG. 4, the lock mechanism 550 locks the guide member 300 inserted through the opening portion 530 at different two places in the central axis C5 direction of the insertion opening portion 36a so that the part 303 (the first bent pressure-contact part 305) of the guide member 300 maintains the bending state. In other words, the lock mechanism 550 locks the held part 307 which has been inserted through the lock mechanism 550 at two places different in height position so that the bending state and the pressure contact state are maintained. The lock mechanism 550 locks the guide member 300 inserted through the opening portion 530 so that the guide member 300 maintains the bending state and the pressure contact state.

As shown in FIG. 1B, FIG. 1C, FIG. 3F, and FIG. 4, the lock mechanism 550 has the lock/introduction portion 551 which is provided in an inner circumferential surface of a first groove portion 573 continuous with the opening portion 530 and which functions as a slide surface for the held part 307 of the part 303 to slide on and which slides and introduces the held part 307 of the part 303. The lock mechanism 550 has a first lack portion 553 which is provided in one part of the lock/introduction portion 551 and which locks the second bent pressure-contact part 307a of the held part 307 that is a part of the part 303. The lock mechanism 550 has a second lock portion 555 which is provided in a second groove portion 575 continuous with the first groove portion 573 and which is provided at a position farther than the first lock portion 553 in the radial direction C7 of the central axis C5 of the insertion opening portion 36a. The second lock portion 555 locks the pressure contact part 307b of the held part 307 which is another part of the part 303. The first groove portion 573 and the second groove portion 575 are formed by cutting out the thick portion of the holding mechanism 500 in the central axis C5 direction of the insertion opening portion 36a (the height direction of the holding mechanism 500). That is, the first groove portion 573 and the second groove portion 575 pierce the thick portion of the holding mechanism 500 in the central axis C5 direction of the insertion opening portion 36a (the height direction of the holding mechanism 500). The first groove portion 573 and the second groove portion 575 are provided at the same height position relative to each other. The second lock portion 555 is provided at a position higher than the first lock portion 553 in the height direction of the holding mechanism 500.

A central axis direction of the first groove portion 573 is provided, for example, along the central axis C5 direction of the insertion opening portion 36a. Specifically, the inner circumferential surface of the first groove portion 573 is provided along the central axis C5 direction of the insertion opening portion 36a. The central axis direction of the first groove portion 573 (the inner circumferential surface of the first groove portion 573) may be inclined relative to the central axis C5 direction of the insertion opening portion 36a. The central axis of the first groove portion 573 is shifted to the central axis C5 of the insertion opening portion 36a in the first diametrical direction R1 of the insertion opening portion 36a. The inner circumferential surface of the first groove portion 573 is, for example, smooth.

The contents described above also apply to the second groove portion 575.

The second groove portion 575 is provided between the first groove portion 573 and the grasp portion 33 in the first diametrical direction R1 of the insertion opening portion 36a, and is provided farther than the first groove portion 573 when the second groove portion 575 is seen from the forceps plug portion 36. The second groove portion 575 has, for example, one of a V-shape, a rectangular shape, a trapezoidal shape, and a parabolic shape. The second groove portion 575 is preferably tapered as it leaves the insertion opening portion 36a. In other words, the second groove portion 575 is preferably tapered in a direction in which the restoring force is applied to the pressure contact part 307b as will be described later, i.e., in a direction of restoration. A shape of the second groove portion 575 is not specifically limited. The second groove portion 575 has a diameter which is substantially the same as a diameter of the guide member 300 so that the guide member 300 having the diameter of, for example, 1 mm fits in the second groove portion 575.

As shown in FIG. 2D and FIG. 3E, the held part 307 slides on the lock/introduction portion 551. The held part 307 slides on the lock/introduction portion 551 so that the lock/introduction portion 551 introduces the held part 307 into the first lock portion 553.

As shown in FIG. 1B, FIG. 1C, and FIG. 2A, the lock/introduction portion 551 has, for example, a J-shape. The lock/introduction portion 551 has a first guide surface portion 551a, a second guide surface portion 551b, and a third guide surface portion 551c. The order in the path of the guide member 300 in the lock/introduction portion 551 is the first guide surface portion 551a, the second guide surface portion 551b, and the third guide surface portion 551c. The first guide surface portion 551a is provided adjacent to the opening portion 530. A part of the second guide surface portion 551b is continuous with the first guide surface portion 551a. The second guide surface portion 551b faces the opening portion 530, and the first lock portion 553 is provided in the second guide surface portion 551b. A part of the third guide surface portion 551c is continuous with another part of the second guide surface portion 551b. Another part of the third guide surface portion 551c is continuous with the second groove portion 575, and guides the held part 307 of the part 303 to the second lock portion 555. The first guide surface portion 551a, the second guide surface portion 551b, and the third guide surface portion 551c are provided, for example, in the central axis C5 direction of the insertion opening portion 36a. The first guide surface portion 551a, the second guide surface portion 551b, and the third guide surface portion 551c may be inclined relative to the central axis C5 direction of the insertion opening portion 36a.

As shown in FIG. 2A, the first guide surface portion 551a is provided beside the opening portion 530. The first guide surface portion 551a is inclined relative to the first diametrical direction R1 of the insertion opening portion 36a. The first guide surface portion 551a faces a end portion of the introduction portion 513, and is provided parallel to the introduction portion 513. Thus, the opening portion 530 intervenes between the first guide surface portion 551a and the end portion of the introduction portion 513. Although described later in detail, the second guide surface portion 551b is provided along the second diametrical direction R2. The second guide surface portion 551b is provided farther than the forceps plug portion 36 when the holding mechanism 500 is seen from the forceps plug portion 36 toward the grasp portion 33. The thick portion of the holding mechanism 500 intervenes between the second guide surface portion 551b and the forceps plug portion 36. The third guide surface portion 551c is inclined relative to the first diametrical direction R1 of the insertion opening portion 36a, and is longer than the first guide surface portion 551a. A inclination angle of the third guide surface portion 551c to the first diametrical direction R1 is smaller than a inclination angle of the first guide surface portion 551a to the first diametrical direction R1.

As shown in FIG. 2A, a length L1 of the thick portion of the holding mechanism 500 provided around the first guide surface portion 551a is adjusted to ensure a pull-down amount in which the part 303 is pulled down toward the grasp portion 33 in a state where the bending state and the pressure contact state are maintained.

As shown in FIG. 1B and FIG. 4, the first lock portion 553 is provided at a position where the second bent pressure-contact part 307a of the guide member 300 extending from the insertion opening portion 36a is bent and locked so that the first bent pressure-contact part 305 of the guide member 300 that is bent may extend in a straight state along the central axis C5 direction of the insertion opening portion 36a. In other words, the first lock portion 553 is provided on the direction side where the first bent pressure-contact part 305 in the bending state and the pressure contact state is restored to the straight state. When the first bent pressure-contact part 305 will be restored to the straight state by the restoring force, the second bent pressure-contact part 307a is pressed against the first lock portion 553 by the restoring force. A first direction which is produced by this pressing is the aforementioned direction of restoration, is a direction toward the central axis C5 of the insertion opening portion 36a, and is a direction to leave the grasp portion 33. In this instance, the second bent pressure-contact part 307a is bent by the first lock portion 553 (the lower edge portion of the first groove portion 573), and brought into pressure contact with the first lock portion 553. Thus, the first lock portion 553 locks the second bent pressure-contact part 307a which is a part of the held part 307. The first lock portion 553 regulates an action of the first bent pressure-contact part 305 in the bending state to be restored to the straight state by the restoring force (resiliency) of the guide member 300. The first lock portion 553 prevents the second bent pressure-contact part 307a inserted in the first groove portion 573 from acting upward from the first lock portion 553 in the central axis C5 direction of the insertion opening portion 36a. In other words, the first lock portion 553 functions as a stopper which prevents the second bent pressure-contact part 307a from coming off the first lock portion 553 in the central axis C5 direction of the insertion opening portion 36a and from being restored to the straight state by the restoring force (resiliency).

The first lock portion 553 is provided on the direction side where the first bent pressure-contact part 305 will be deformed by the pull-out of the treatment tool 400. The first lock portion 553 prevents a deformation of the guide member 300 when the bending state and the pressure contact state will be released by the pull-out of the treatment tool 400.

As shown in FIG. 1B and FIG. 4, the first lock portion 553 is provided at the lower edge portion of the first groove portion 573 which is an edge portion of the first groove portion 573 closer to the insertion opening portion 36a. Specifically, the first lock portion 553 functions as the lower edge portion of the second guide surface portion 551b which is a part of the lock/introduction portion 551. The first lock portion 553 is provided adjacent to the insertion opening portion 36a in the first diametrical direction R1 of the insertion opening portion 36a.

As shown in FIG. 1B and FIG. 4, the second lock portion 555 is provided on the side which is regulated an action that another part (the pressure contact part 307b) of the held part 307 of the part 303 in the bending state by the first lock portion 553 is restored to the straight state. For example, the second lock portion 555 is provided opposite to the first lock portion 553 across the central axis of the first groove portion 573. When the pressure contact part 307b bent by the first lock portion 553 will be restored to the straight state by the restoring force, the pressure contact part 307b is pressed against the second lock portion 555 by the restoring force. A second direction which is produced by this pressing is opposite to the first direction, is a direction to leave the central axis C5 of the insertion opening portion 36a, and is a direction toward the grasp portion 33 side. In this instance, the pressure contact part 307b is brought into pressure contact with the second lock portion 555 (the upper edge portion of the second groove portion 575) by the restoring force. Thus, the second lock portion 555 locks the pressure contact part 307b. The second lock portion 555 regulates the action of the pressure contact part 307b in the bending state to be restored to the straight state by the restoring force (resiliency) of the guide member 300. The second lock portion 555 prevents the pressure contact part 307b inserted in the second groove portion 575 from acting upward from the second lock portion 555 in the central axis C5 direction of the insertion opening portion 36a. In other words, the second lock portion 555 functions as a stopper which prevents the pressure contact part 307b inserted in the second groove portion 575 from coming off the second lock portion 555 in the central axis C5 direction of the insertion opening portion 36a and from being restored to the straight state by the restoring force (resiliency).

As shown in FIG. 1B and FIG. 4, the second lock portion 555 is provided at a position higher than the first lock portion 553. For example, the second lock portion 555 is provided at the upper edge portion of the second groove portion 575 which is an edge portion of the second groove portion 575 farther from the insertion opening portion 36a than the first lock portion 553. The second lock portion 555 is provided farther than the first lock portion 553 when the holding mechanism 500 is seen from the forceps plug portion 36 toward the grasp portion 33. Thus, the second lock portion 555 is provided farther from the insertion opening portion 36a than the first lock portion 553. The second lock portion 555 is provided collinearly with the central axis C5 of the insertion opening portion 36a and the first lock portion 553.

The lock mechanism 550 functions as a catch portion such that the held part 307 introduced by the lock/introduction portion 551 is caught on the lock mechanism 550. Specifically, the part 303 is locked by the first lock portion 553 and the second lock portion 555 so that the part 303 is sheared by the first lock portion 553 and the second lock portion 555.

[Function]

How the guide member 300 guides the treatment tool 400 into a duodenal papilla is described below by way of example.

[Guide Work for Treatment Tool 400]

The guide member 300 is engaged with, for example, an unshown groove portion of the treatment tool 400. The groove portion is provided, for example, over the entire length of the treatment tool 400 along the axial direction of the treatment tool 400. The proximal end portion 301 of the guide member 300 is out of the groove portion, and is separated from the treatment tool 400. That is, the distal end sides of the guide member 300 and the treatment tool 400 are integral, but the proximal sides thereof are separate from each other as shown in FIG. 1B.

In this state, the guide member 300 and the treatment tool 400 are inserted into the insertion device 10 from the insertion opening portion 36a via the forceps plug portion 36 and the treatment tool insertion opening portion 35a. The guide member 300 and the treatment tool 400 are inserted through the treatment tool insertion channel and then protrude to the lateral side of the distal end portion of the insertion portion 20 from the distal opening portion. The distal end portion of the guide member 300 then reaches the subject such as the bile duct, and guides the treatment tool 400 to the subject. The proximal end portion 301 of the guide member 300 and the proximal end portion of the treatment tool 400 are exposed to the outside of the insertion device 10 from the insertion opening portion 36a.

In the above, the guide member 300 and the treatment tool 400 are inserted by the operation with the right hand of the surgeon in a state where the grasp portion 33 is grasped with the left hand of the surgeon.

[Bending/Pressure Contact Work in Holding Work]

In this state, at the proximal end portion 301 of the guide member 300 exposed from the groove portion and to the outside of the insertion device 10, the part 303 is held by the holding unit 501.

Specifically, as indicated by solid lines in FIG. 2B and FIG. 3B, the part 303 is bent, for example, in an L-shape relative to the insertion opening portion 36a, and is pulled to the grasp portion 33 side from the forceps plug portion 36 side. Accordingly, the part 303 is brought into pressure contact with the inner circumferential surface and upper-edge-side inner circumferential edge of the insertion opening portion 36a (the forceps plug portion 36), and brought into pressure contact with the upper edge surface from the upper part of the insertion opening portion 36a (the forceps plug portion 36) toward the upper edge surface of the forceps plug portion 36. The first bent pressure-contact part 305 is then formed, and the bending state and the pressure contact state are maintained.

If the first bent pressure-contact part 305 is formed and the bending state and the pressure contact state are maintained, the guide member 300 does not necessarily need to be pulled.

The bending/pressure contact work may be omitted, and the first bent pressure-contact part 305 may be formed during later-described guide work or lock work.

[Guide Work and Lock Work in Holding Work]

As shown in FIG. 2C and FIG. 3C, the guide portion 510 guides the held part 307 and the part of the extending part 309 that are in contact with the guide portion 510 toward the lock mechanism 550 in a state where the first bent pressure-contact part 305 is formed.

Specifically, as shown in FIG. 2B and FIG. 3B, the held part 307 and the part of the extending part 309 contact the contact portion 511 which is exposed to the outside. In this instance, the held part 307 and the part of the extending part 309 are bent toward the grasp portion 33 in the central axis C5 direction of the insertion opening portion 36a. That is, the held part 307 and the part of the extending part 309 are pulled down toward the grasp portion 33. The thick portion of the holding mechanism 500 provided around the first guide surface portion 551a prevents the held part 307 and the part of the extending part 309 from being directly inserted into the first groove portion 573 in the lock mechanism 550.

As shown in FIG. 2C and FIG. 3C, the held part 307 and the part of the extending part 309 slide on the contact portion 511 toward the introduction portion 513, and contact the introduction portion 513. In this instance, the first bent pressure-contact part 305 moves in the gap portion 571 in the second diametrical direction R2. As shown in FIG. 3D, the held part 307 and the part of the extending part 309 rise along the introduction portion 513, and are provided along the central axis C5 of the insertion opening portion 36a. That is, the held part 307 and the part of the extending part 309 are pulled and raised.

As shown in FIG. 3D, the held part 307 is pulled and raised toward the forceps plug portion 36 side, thereby sliding on the introduction portion 513, being inserted through the opening portion 530, and being inserted into the lock mechanism 550.

As shown in FIG. 3E and FIG. 3F, in the lock mechanism 550, the held part 307 is introduced into the first lock portion 553 and the second lock portion 555 by the lock/introduction portion 551. Specifically, the held part 307 slides in the first guide surface portion 551a, the second guide surface portion 551b, and the third guide surface portion 551c of the lock/introduction portion 551 in order, and is inserted into the second groove portion 575.

The held part 307 is then locked by the first lock portion 553 in the second guide surface portion 551b and by the second lock portion 555 in the second groove portion 575.

As shown in FIG. 4, in the first lock portion 553, when the first bent pressure-contact part 305 will be restored to the straight state by the restoring force, a part (the second bent pressure-contact part 307a) of the held part 307 is pressed against the lower edge portion of the first groove portion 573 which functions as the first lock portion 553 by the restoring force. In this instance, the second bent pressure-contact part 307a is bent by the first lock portion 553, and brought into pressure contact with the first lock portion 553. Accordingly, the first lock portion 553 locks the second bent pressure-contact part 307a.

As shown in FIG. 4, in the second lock portion 555, when the held part 307 bent by the first lock portion 553 will be restored to the straight state by the restoring force, another part (the pressure contact part 307b) of the held part 307 is pressed against the lower edge portion of the second groove portion 575 which functions as the second lock portion 555 by the restoring force. The aforementioned second direction of the pressing in the second lock portion 555 is opposite to the aforementioned first direction of the pressing in the first lock portion 553. In this instance, the pressure contact part 307b is brought into pressure contact with the second lock portion 555 by the restoring force. Accordingly, the second lock portion 555 locks the pressure contact part 307b. The proximal end portion 301 of the held part 307 engages with the second lock portion 555. The second lock portion 555 thereby locks the proximal end portion 301 of the held part 307.

The first lock portion 553 locks the second bent pressure-contact part 307a. The second lock portion 555 locks the pressure contact part 307b. The second lock portion 555 is provided at a position higher than the first lock portion 553 in the central axis C5 direction of the insertion opening portion 36a, and is provided farther from the insertion opening portion 36a than the first lock portion 553. The second lock portion 555 is provided substantially collinearly with the first lock portion 553. Thus, the first lock portion 553 and the second lock portion 555 lock the held part 307 so that the first lock portion 553 and the second lock portion 555 shear the held part 307 by the mutual positional relation.

In this way, the guide member 300 is locked at a total of three places: the first bent pressure-contact part 305, the second bent pressure-contact part 307a which is a part of the held part 307, and the pressure contact part 307b which is another part of the held part 307. The locking places are height positions different from one another.

The first bent pressure-contact part 305 may be caught on the edge portion of the contact portion 511 adjacent to the forceps plug portion 36 in the central axis C5 direction of the insertion opening portion 36a. This reinforces the lock.

[Replacement Work and Lock release Work for Treatment Tool 400]

When the treatment tool 400 is replaced with another treatment tool 400, the treatment tool 400 alone is pulled so that the treatment tool 400 may be peeled off the guide member 300. The treatment tool 400 is then pulled out of the insertion device 10.

After the treatment tool 400 is detached from the insertion device 10, the held part 307 is temporarily detached from the holding mechanism 500 by a procedure reverse to the procedure described above. Thus, the held part 307 only moves these parts along the lock/introduction portion 551.

In the lock release work in the held part 307, the held part 307 only slides on the lock/introduction portion 551.

In this state, the distal end portion of the guide member 300 remains protruding from the distal opening portion.

Another treatment tool 400 having a monorail portion is then inserted into the insertion device 10 from the insertion opening portion 36a along the guide member 300 via the monorail portion. This treatment tool 400 then protrudes from the distal opening portion along the guide member 300, and reaches the subject.

In this way, the replaced treatment tool 400 is guided to the subject by the guide member 300.

After the treatment tool 400 is guided, the part 303 is again held by the holding unit 501 in the same manner as above as shown in FIG. 3A and FIG. 3B at the exposed proximal end portion 301 of the guide member 300.

In the replacement work and the lock release work, the guide member 300 and the treatment tool 400 are operated with the left hand of the surgeon in a state where the grasp portion 33 is grasped with the right hand of the surgeon.

[Prevention of Deformation of First Bent Pressure-Contact Part 305 Involving Replacement Work]

If the treatment tool 400 is pulled away from the grasp portion 33 in the first diametrical direction R1 of the insertion opening portion 36a for replacement as described above, a pull force produced by the pulling acts on the first bent pressure-contact part 305 in the central axis C5 of the insertion opening portion 36a. This action releases the bending state and the pressure contact state, the first bent pressure-contact part 305 is released and deformed, and an unshown loop portion will be formed in the part of the guide member 300 immediately after exposed from the forceps plug portion 36. The deformation including the formation of this loop portion is caused when the treatment tool 400 inserted in the insertion device 10 is pulled out by the pull force. In this case, the proximal end portion side of the guide member 300 including the held part 307 is positioned and fixed in the holding mechanism 500 by the holding mechanism 500, but the distal end portion of the guide member 300 may be displaced due to the deformation. That is, displacement occurs at the distal end portion; for example, the distal end portion withdraws into the insertion device 10 from the distal opening portion.

However, the first lock portion 553 is provided on the direction side where the first bent pressure-contact part 305 will be deformed by the pull-out of the treatment tool 400. Specifically, the first lock portion 553 is provided at the lower edge portion of the first groove portion 573, and functions as the lower end portion of the second guide surface portion 551b. The first lock portion 553 is provided adjacent to the insertion opening portion 36a, and is provided at a position higher than the insertion opening portion 36a. Therefore, even if the treatment tool 400 is pulled out, the operation of the first bent pressure-contact part 305 is prevented, and the bending state and the pressure contact state are maintained. Even if the treatment tool 400 is pulled out, the deformation of the first bent pressure-contact part 305 is prevented, and the formation of the loop portion is prevented in the first bent pressure-contact part 305. That is, in the case of the pull force alone, deformation is prevented, the formation of the loop portion is prevented, and the bending state and the pressure contact state are maintained.

The formation of the loop portion is also prevented by the positioning and fixing at the aforementioned three places: the first bent pressure-contact part 305, the second bent pressure-contact part 307a, and the pressure contact part 307b.

This prevents the occurrence of displacement at the distal end portion of the guide member 300.

### Advantageous Effects

Thus, in the present embodiment, the part 303 is inserted through the opening portion 530 and inserted into the lock mechanism 550 in a state where the first bent pressure-contact part 305 is formed. The part 303 is then locked at two places different from each other in height position in the lock mechanism 550 which is provided on the radial direction C7 of the central axis C5 of the insertion opening portion 36a adjacent to the insertion opening portion 36a at a position higher than the insertion opening portion 36a. Thus, in the present embodiment, the guide member 300 can be easily and stably fixed.

In the present embodiment, the part 303 can be certainly locked by the lock/introduction portion 551.

In the present embodiment, the first lock portion 553 is provided at the lower edge portion of the first groove portion 573. The second lock portion 555 is provided at the upper edge portion of the second groove portion 575, provided farther from the insertion opening portion 36a than the first lock portion 553, and provided substantially collinearly with the first lock portion 553. Thus, in the present embodiment, the configuration of the holding mechanism 500 can be simpler. The part 303 is locked by the first lock portion 553 and the second lock portion 555 so that the part 303 is sheared by the first lock portion 553 and the second lock portion 555. Thus, in the present embodiment, the part 303 can be certainly locked by forces in opposite directions at two places.

In the present embodiment, the part 303 can be certainly introduced into the second lock portion 555 by the lock/introduction portion 551.

In the present embodiment, the holding mechanism 500 is provided adjacent to the insertion opening portion 36a, specifically, provided in the operation portion 30. Thus, in the present embodiment, the first bent pressure-contact part 305 can be continuous with and adjacent to the held part 307, the bending state and the pressure contact state can be maintained, and the guide member 300 can be positioned and fixed at three places: the first bent pressure-contact part 305, the second bent pressure-contact part 307a, and the pressure contact part 307b.

The holding mechanism 500 is integral with the operation portion 30 or is a separate body attachable to and detachable from the operation portion 30. When the holding mechanism 500 is integral with the operation portion 30, the holding mechanism 500 can be washed together with the operation portion 30. When the holding mechanism 500 is separate from the operation portion 30, the holding mechanism 500 can adapt to the treatment tools 400 having various thickness.

The forceps plug portion 36 may be either a reusable type or a disposable type. The reusable type forceps plug portion 36 is harder than the disposable type forceps plug portion 36.

[Modification 1 of Holding Mechanism 500]

Although the holding mechanism 500 is provided in the operation portion 30 in the present embodiment, the holding mechanism 500 does not need to be limited thereto if the holding mechanism 500 is provided adjacent to the insertion opening portion 36a. For example, the holding mechanism 500 may be provided in the forceps plug portion 36.

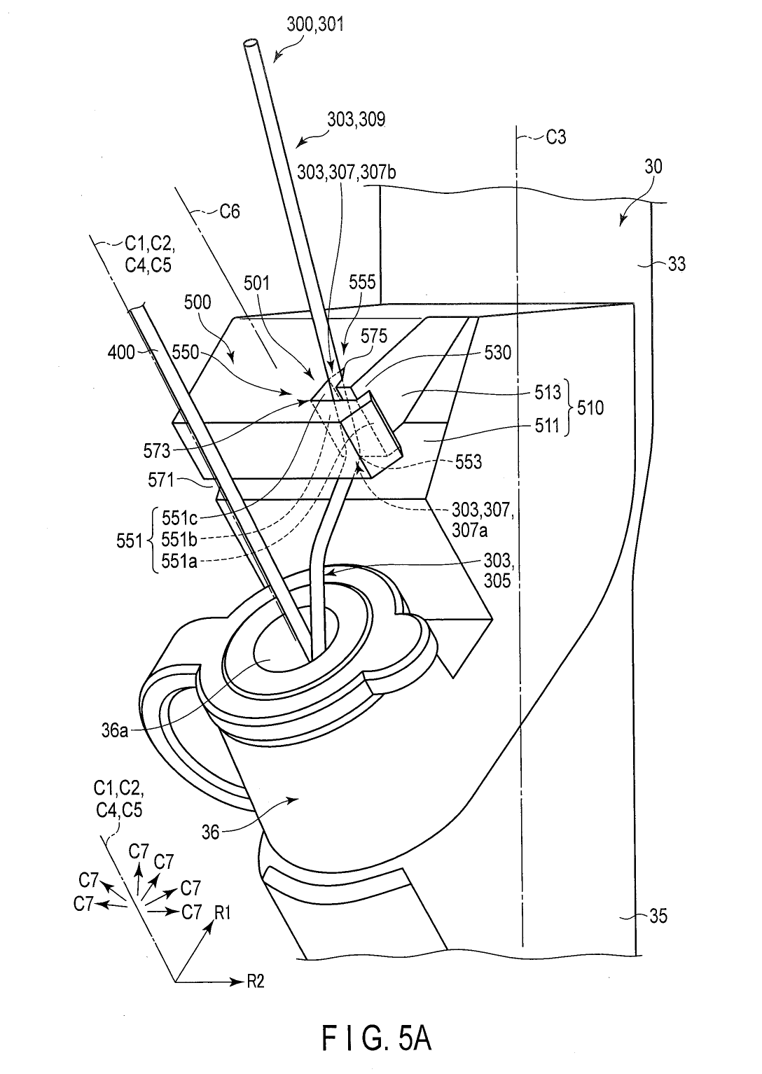

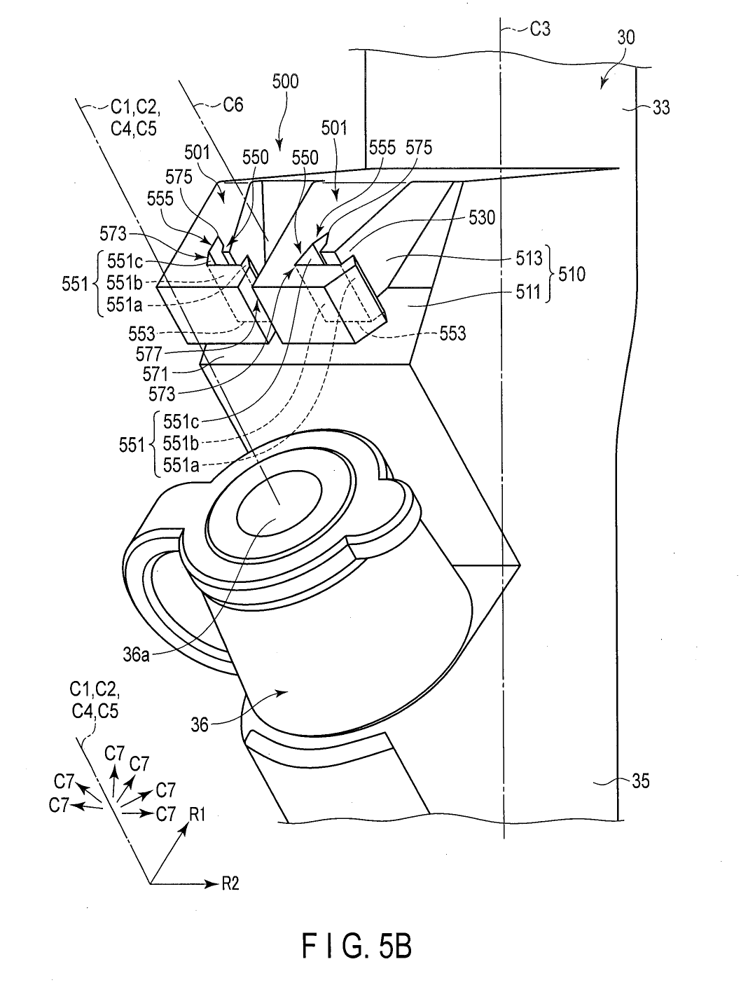

In this case, the holding mechanism 500 is integral with the forceps plug portion 36 as shown in FIG. 5A, or is a separate body attachable to and detachable from the forceps plug portion 36, which is, however, not shown.

When the holding mechanism 500 is integral with the operation portion 30, for example, the holding mechanism 500 is provided in the insertion device 10 so that the holding mechanism 500 is provided at the position shown in the embodiment. Such a holding mechanism 500 is insert-molded together with the forceps plug portion 36.

When the holding mechanism 500 is separate from the forceps plug portion 36, the holding mechanism 500 is detachably attached to, for example, the forceps plug portion 36. In this case, for example, the holding mechanism 500 has a belt-shaped member which is wound around the forceps plug portion 36, and is detachably attached to the forceps plug portion 36 by the belt-shaped member.

Consequently, the holding mechanism 500 can be freely attached to and detached from the insertion device 10 together with the forceps plug portion 36. The holding mechanism 500 is disposable together with the forceps plug portion 36.

[Modification 2 of Holding Mechanism 500]

The holding mechanism 500 has only to have at least one holding unit 501. As shown in FIG. 5B, when more than one holding unit 501 are provided, more than one guide member 300 are held by the respective holding units 501. The holding units 501 are provided side by side, for example, along the second diametrical direction R2 so that one holding unit 501 is provided on a left side of the holding mechanism 500 in the second diametrical direction R2 and the other holding unit 501 is provided in a center of the holding mechanism 500 in the second diametrical direction R2.

The contact portion 511 and the gap portion 571 are shared by the two holding units 501, and are parts common to the two holding units 501. The units having the introduction portions 513, the openings 530, the lock/introduction portions 551, the first lock portions 553, and the second lock portions 555 are collinearly provided in the second diametrical direction R2, and are provided adjacent to each other. The introduction portions 513 are continuous with each other, and the thick portion of the holding mechanism 500 intervenes between the units.

In this state, the order in the path to hold the guide members 300 is the contact portions 511, the introduction portions 513, the openings 530, the lock/introduction portions 551, the first lock portions 553, and the second lock portions 555. The contact portions 511, the introduction portions 513, and the lock/introduction portions 551 are continuous peripheral surfaces of the holding mechanism 500, and the guide members 300 slide on these parts. In other words, the guide members 300 move in these parts along these parts. Each part is described below.

An opening portion 577 through which the part 303 is inserted and pulled out is provided between the holding unit 501 provided in the center of the holding mechanism 500 and the holding unit 501 provided on the left side of the holding mechanism 500 in the second diametrical direction R2. The opening portion 577 functions as a communication path for the part 303 to be provided in the contact portion 511 of the holding unit 501 provided in the center of the holding mechanism 500. In this case, the opening portion 577 is tapered toward the grasp portion 33 from the forceps plug portion 36 in the first diametrical direction R1 of the insertion opening portion 36a, and functions as a guide portion.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A holding mechanism which holds a guide member to guide a treatment tool that is inserted into a lumen through an insertion portion of an insertion device to be inserted into the lumen, the holding mechanism comprising:
an opening portion through which the guide member is inserted in a bending state where one part of the guide member exposed to an outside of the insertion device from an insertion opening portion of the insertion device through which the guide member is inserted is bent relative to the insertion opening portion;
a gap portion which has a cutout surface that is provided by cutting out one part of the holding mechanism in a radial direction of a central axis of the insertion opening portion, and that guides the guide member into the opening portion from the insertion opening portion; and
a lock mechanism which is provided in the radial direction and which locks the guide member inserted through the opening portion at two places different from each other in positions in the central axis direction of the insertion opening portion so that the one part maintains the bending state.

2. The holding mechanism according to claim 1, wherein the lock mechanism is provided at a position farther from the insertion opening portion in the central axis direction of the insertion opening portion.

3. The holding mechanism according to claim 1, wherein a central axis direction of the lock mechanism is parallel to the central axis direction of the insertion opening portion.

4. The holding mechanism according to claim 1, wherein the lock mechanism comprises;
a lock/introduction portion which is provided in a first groove portion continuous with the opening portion and which slides and introduces the guide member,
a first lock portion which is provided in one part of the lock/introduction portion and which locks one part of the guide member,
a second lock portion which is provided in a second groove portion continuous with the first groove portion and which is provided at a position farther than the first lock portion in the radial direction and which locks another part of the guide member.

5. The holding mechanism according to claim 4, wherein the first lock portion is provided at a position where the guide member extending from the insertion opening portion is bent and locked so that the guide member that is bent extends in a straight state along the central axis direction of the insertion opening portion, and
the second lock portion is provided on the side which is regulated an action that the guide member bent by the first lock portion is restored to the straight state.

6. The holding mechanism according to claim 5, wherein the first groove portion and the second groove portion pierce the holding mechanism in the central axis direction of the insertion opening portion,
the first lock portion is provided at an edge portion of the first groove portion closer to the insertion opening portion,
the second lock portion is provided at an edge portion of the second groove portion farther from the insertion opening portion than the first lock portion, and
the guide member is locked by the first lock portion and the second lock portion so that the guide member is sheared by the first lock portion and the second lock portion.

7. The holding mechanism according to claim 4, wherein the lock/introduction portion comprises;
a first guide surface portion provided adjacent to the opening portion,
a second guide surface portion which is continuous with the first guide surface portion and which faces the opening portion and in which the first lock portion is provided, and
a third guide surface portion which is continuous with the second groove portion and which guides the guide member to the second lock portion.

8. The holding mechanism according to claim 1, wherein the guide member is inserted through the opening portion in a pressure contact state where the one part exposed to the outside from the insertion opening portion is in pressure contact with the insertion opening portion, and
the lock mechanism locks the guide member inserted through the opening portion so that the guide member maintains the bending state and the pressure contact state.

9. The holding mechanism according to claim 1, wherein more than one holding unit having the opening portion and the lock mechanism are provided, and
more than one guide member are held by the respective holding units.

10. The holding mechanism according to claim 1, wherein the insertion device comprises an operation portion which is coupled to a proximal end portion of the insertion portion, and
the holding mechanism is provided in the operation portion having the insertion opening portion, or is provided in a forceps plug portion which is provided in the operation portion and which has the insertion opening portion.

11. The holding mechanism according to claim 10, wherein the holding mechanism is integral with the operation portion or is a separate body attachable to and detachable from the operation portion, or is integral with the forceps plug portion or is a separate body attachable to and detachable from the forceps plug portion.

12. An insertion device comprising the holding mechanism according to claim 1.
